(19) United States
(12) Patent Application Publication
(10) Pub. No.: US 2005/0212239 A1
(43) Pub. Date: Sep. 29, 2005

(54) UVC transport cart

(75) Inventor: Thomas Eric Carter, Maplewood, NJ (US)

Correspondence Address: FITZPATRICK CELLA HARPER & SCINTO, 30 ROCKEFELLER PLAZA, NEW YORK, NY, 10112, US

(73) Assignee: Caddy Corporation, Bridgeport, NJ (US)

(21) Appl. No.: 10/808,178

(22) Filed: Mar. 24, 2004

(51) Int. Cl. B62B 3/00

(52) U.S. Cl. 280/47.35

(57) ABSTRACT

A food service transport cart includes a mobile housing having at least one door containing ports for at least one food service tray therein. A UV light source is mounted in the housing and connected to a source of power. A switch and timer are provided for controlling the source of power to activate the light source for a predetermined period of time after the door is closed.

### BACKGROUND OF THE INVENTION

1. Field of the Invention

The present invention relates to a food service transport cart, more in particular, to a cart utilizing ultraviolet irradiation for disinfecting the interior of the cart each time the doors to the cart are opened.

2. Background of the Invention

In hospitals and other institutions, or places of public accommodation which require food prepared in a central location to be transported directly to the patient or guest, various types of transportation carts are used. Typically, these carts have one or two doors and a food tray support system contained therein for holding one or more trays of food to be delivered to one or more individuals or sites within the particular building involved. These carts are designed to hold hot, warm or cold foods in an insulated environment during the typical delivery time period. In some cases the carts are simply reused, without cleansing, and in others, particularly in health care facilities, the carts may be washed or cleaned between delivery periods. However, they may be used multiple times within a particular delivery period.

It has been found, particularly within hospitals or buildings of public accommodations or the like, that bacteria or viruses in the air may enter the cart during a particular delivery, and remain in the cart during further deliveries. This results because the surrounding air in the building is allowed to enter the inner holding chamber of the cart. As a result, food, utensils and dishware in the cart to be delivered to subsequent patients may become contaminated with undesirable bacteria and viruses and create an unnecessary health hazard.

Although it has previously been proposed to provide ultraviolet irradiation in particular food storage stationary facilities, such as, for example, certain refrigeration units or microwave ovens, no system has previously been developed utilizing ultraviolet radiation, a known and successful method of disinfecting spaces in a food service cart so that the items in the cart are continuously disinfected while the cart is in use and its doors repeatedly opened and close.

### OBJECTIVE OF THE INVENTION

It is an object of the present invention to provide an improved food service cart with a self-contained air disinfection system.

Another object of the invention is to provide an ultraviolet irradiation device for a food service cart which is relatively simply in construction and adapted for use in a portable cart.

A further object of the present invention is to provide a mobile food service cart self-contained irradiation unit, selectively operable for predetermined periods of time after the door or doors of the cart are opened.

### BRIEF SUMMARY OF THE INVENTION

In accordance with an aspect of the present invention, a food service transport cart is provided which includes a mobile housing, preferably mounted on wheels, that includes at least one door which provides selective access to the interior of the housing. The housing contains a tray support system, such as, for example, molded support flanges on its side walls and a central wall, which are adapted to support a plurality of food service trays in one or more stacks with the trays spaced from one another to allow room therebetween for the food, utensils and dishware on the tray.

At least one UV-C irradiation lamp is positioned in the housing, for example, on the interior surface of the front door or on the back wall, in order to expose the air and items in the housing to the disinfection effects of the ultraviolet light.

A power source is mounted in the housing for selectively supplying electrical power to the light source. This power source consists of a rechargeable battery pack for supplying electrical power or current to the UV-C lamp. One or more switches are positioned in the cart and connected between the power source and the lamp or lamps, to allow power to flow from the battery pack to the lamp when the door is closed. These switches operate on contact with the closed door, and are in turn connected to a timer which will cut off the power supply to the lamp or lamps after a predetermined period of time following the closure of the door. If the doors are opened before that predetermined period of time passes, the light is automatically shut off when the door moves away from the contact switches. This will automatically reset the timer.

The light sources used in the present invention are shaped similarly to fluorescent tubular light bulbs, but produce ultraviolet light. Such light sources or lamps produce UV-C irradiation at a wavelength of about 254 nm, with an intensity of about 142 μw/cm². Preferably, the lights are positioned in a vertical direction and have a length sufficient to cover substantially the entire interior height of the enclosure so that all of the trays and air spaces between them are exposed directly to UV light.

The disinfection unit of the present invention will be able to disinfect the cabinet air and interior inactivate common bacteria found in hospital and other institutional air, such as escherichia coli, pseudomonas, fluoresces, serratia marcescen, Micrococcus, luteus and bacillus subtilis.

The battery pack used in the food service carts of the present invention preferably are rechargeable so that each time the cart is put in storage, for example, between service delivery periods, it may be plugged into a wall to recharge the battery.

The above, and other objects, features, and advantages of this invention will be apparent in the following detailed description of illustrative embodiments thereof, which is to be read in connection with the accompanying drawings wherein:

### BRIEF DESCRIPTION OF THE DRAWINGS

FIG. 1 is a perspective view of a food service cart constructed in accordance with the present invention;

FIG. 2 is a sectional view taken along line 2-2 of FIG. 1;

FIG. 3 is a sectional view taken along line 3-3 of FIG. 2;

FIG. 4 is a sectional view similar to FIG. 2 of another embodiment of the invention;

FIG. 5 is a sectional view similar to FIG. 2, with the doors removed, of the embodiment of FIG. 4 showing the interior tray support system and the UV light source mounted on the rear wall of the cart;

### DETAILED DESCRIPTION OF THE PREFERRED EMBODIMENTS

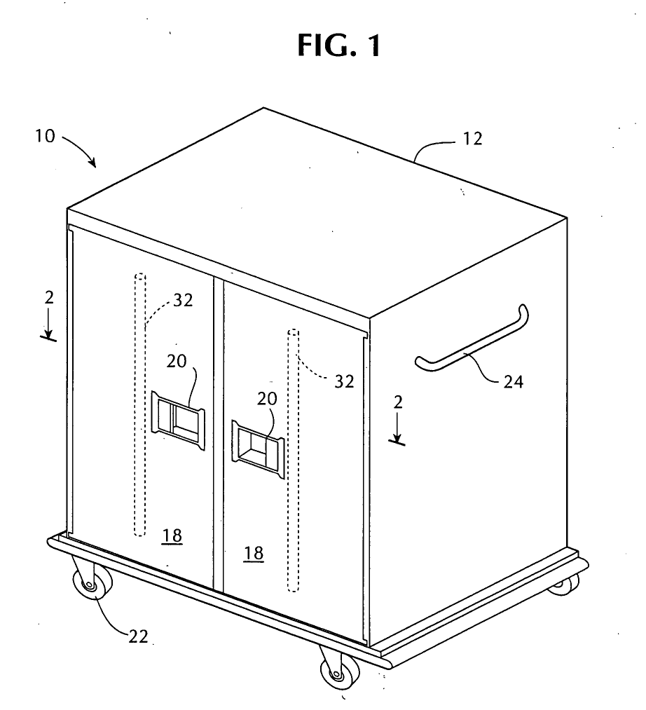

Referring now to drawings in detail, and initially to FIG. 1, a food service transport cart 10, constructed in accordance with the present invention is illustrated.

The cart includes a generally rectangular or other shaped housing 12 of conventional relatively rigid but lightweight construction containing an interior space and an opening 14 in its front wall 16. Preferably, the cart is formed with insulated walls in order to maintain the food placed in the cart for delivery in a relatively warm or cool state.

The opening 14 is closed by one or more doors 18. In the illustrative embodiment, two doors 18 are utilized which are hinged on opposite sides of opening 14 to the side walls of the housing. The doors also are formed of conventional materials, and may have integral handles 20 for selectively opening one or both sides thereof.

The cart is supported on dolly type wheels 22 in a conventional manner so that it can be easily moved through the corridors of the hospital or institution.

Preferably, a handle 24 is provided on one of the side walls of the cart for ease of maneuvering by the operator.

One form of material that could be used to manufacture the housing and doors of the cart is a stressed skin thermoplastic panel material having a foam filled center for insulation. Such panel structure materials are commercially available and need not be described herein in detail.

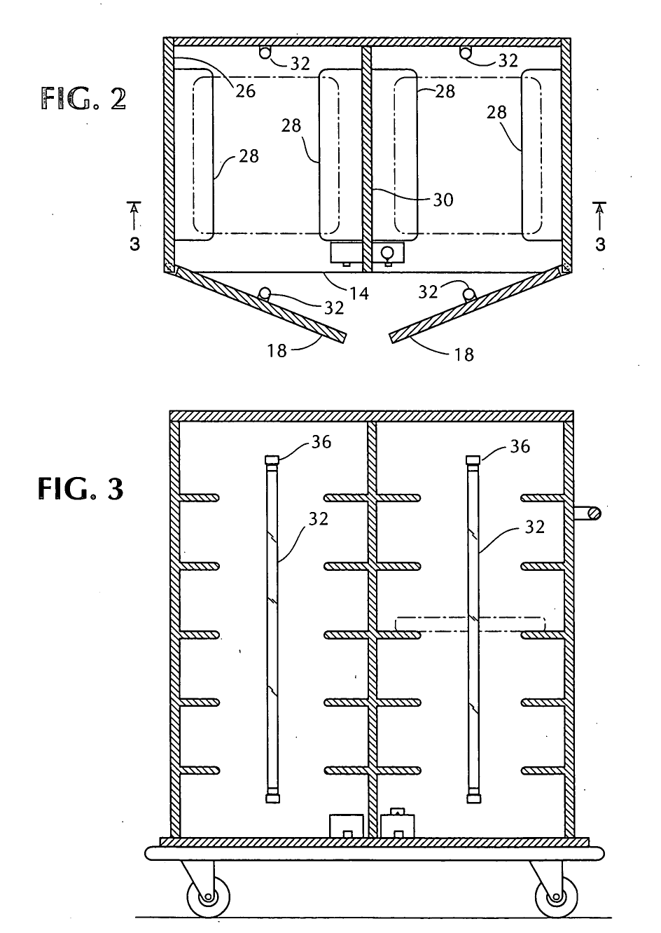

The interior of the transport cart 12 includes a tray support structure which may be of any conventional form currently used in the food service industry. In the illustrative embodiment of the invention, the inner surfaces 26 of the sidewalls of the cart are formed with integral flanges 28 extending therefrom at predetermined vertically spaced intervals. A central support panel 30 is also provided, which has similar support flanges 28 integrally formed therein at the same levels as the flanges 28 on the sidewalls. Thus, these flanges provide support surfaces for food service trays 30, illustrated in dotted lines in FIGS. 2 and 3.

As noted above, in one embodiment of the invention, the UV light sources 32 are mounted on the inner surfaces 34 of the cart door or doors. These UV lights are commercially available in different sizes and need not be described in detail. Their external physical appearance is similar to that of tubular fluorescent bulbs, with contact elements at their ends that are supported in electrical contact sockets 36 in a known manner. Such bulbs may also be mounted on the back wall of the cart.

In the embodiment of the invention illustrated in FIG. 2, the bulbs are mounted on the inner face of the door, and project therefrom. Preferably, in this embodiment the bulbs would be guarded by a protective grid, lens or other transparent barrier (not shown) to avoid inadvertent contact with the trays or other objects which might cause the bulbs to break.

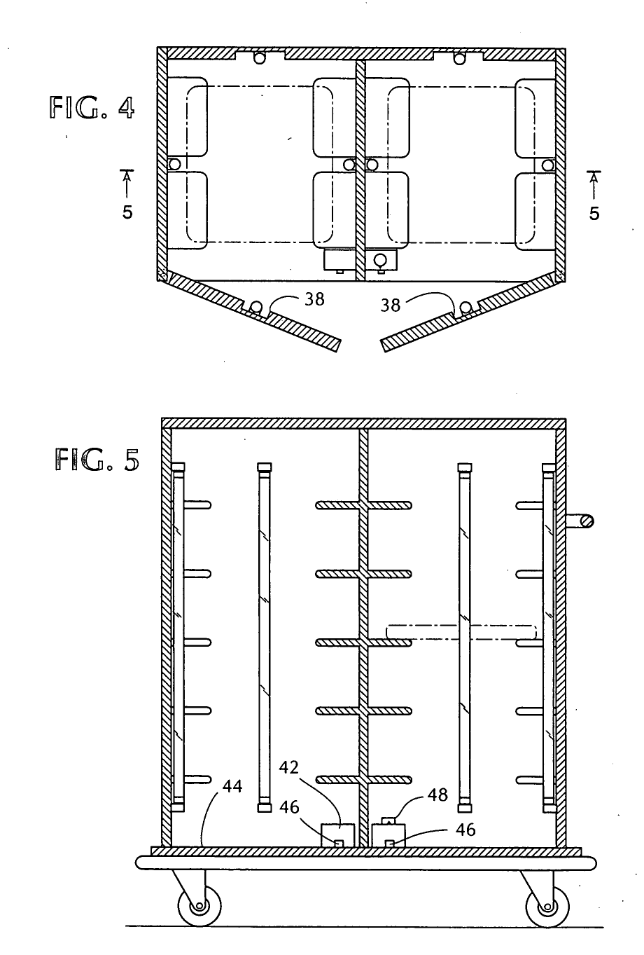

In the embodiment of the invention illustrated in FIG. 4, recesses 38 are formed in the inner surfaces 34 of the doors, and the bulbs 32 and sockets 36 are mounted within the recess. This structure provides further protection for the bulb against inadvertent breakage. However, if desired any form of convenient transparent lens or shielding system to protect the bulb may also be installed over the recesses.

While the above illustrative embodiments of the present invention show a single bulb on each door, the number of bulbs to be utilized can vary depending upon the size of the cart.

The embodiment of the invention illustrated in FIG. 4 also shows the use of UV bulbs 32 mounted within recesses 40 formed in the back wall of the housing. As with the other embodiments, the ends of the bulbs are mounted in conventional sockets 36 appropriate for the bulbs. When bulbs are used on the rear wall the bulbs on the door may optionally be omitted.

In accordance with another feature of the present invention, the UV bulbs are provided with power through electrical connectors (not shown) from a rechargeable battery pack 42 of conventional construction. The battery pack is mounted in or on the cart in any convenient manner. In the illustrative embodiment of the invention shown in FIG. 5, it is mounted on the bottom wall 44 in the center of the cart.

On/off switches 46 are provided in the bottom of the cart in position to contact the door or doors when they are closed. In the illustrative embodiment of the invention shown in FIG. 4, the switches are depicted as being on the bottom wall 44 of the cart adjacent the battery pack. However, they could be mounted on the top wall or the central support member 30 as well. The switches are set so that when the door is closed the switches allow current to pass from the battery pack to the UV bulbs. When the door is opened, the switches open the circuit so that the bulbs are extinguished in order to avoid undesirable exposure to personnel in the area.

Because the space within the housing 12 is limited, it is not necessary for the UV bulbs to be operating at all times. Thus, a timer 48 is provided in conjunction with switches 46 so that after the switches have allowed the UV lamps to operate for a predetermined period of time sufficient to disinfect the interior of the cart, current to the lamps is shut off. This not only preserves the life of the battery pack, but avoids unnecessary exposure of the interior of the cart to the UV light waves. In addition, the timer is selected such that if the doors are opened before the lights have been on for the desired period of time, not only will the switches cause the lights to be shut off, but the timer will reset itself for a new complete timing period to commence once the doors are again closed.

While the UV lamps have been shown on the doors or rear of the interior of the enclosure, they may be located elsewhere within the transport cart as would occur to those skilled in the art. For example, individual horizontal bulbs could be mounted in the recesses in the side walls of the cart between the flanges or support elements 28. Such an arrangement, like the vertical arrangement described above in the illustrated embodiments, assures that all exposed surfaces and all of the air on the interior of the cart are exposed to the UV light each time the doors are opened and closed.

Although illustrative embodiments of the present invention have been described herein in detail, it is to be understood that the invention is not limited to those precise embodiments, and that various changes and modifications can be affected therein without departing from the scope or spirit of this invention.

1. A food service transport cart comprising a mobile housing including at least one door, means in the housing for supporting at least one food service tray; a UV light source mounted in said housing; a source of power for said UVC light source; and means for controlling said source of power for activating said light source when said at least one door is closed.

2. A food service cart as defined in claim 1 wherein said means for controlling said source of power includes switch means which is activated by said door in its closed position to supply power to said UV light source.

3. A food service transport cart as defined in claim 2 wherein said means for controlling said source of power includes timer means for stopping the supply of power to said UV light source after a predetermined time interval following closure of said at least one door.

4. A food service transport cart as defined in claim 3 wherein said UV light source is mounted on the inside of said at least one door.

5. A food service transport cart as defined in claim 3 wherein said UV light source is mounted on at least one inner wall of the cart.

6. A food service transport cart as defined in claim 3 wherein said source of power is a battery pack.

7. A food service transport cart as defined in claim 3 wherein said source of power is a rechargeable battery pack.

8. A food service cart as defined in claim 3 wherein said cart includes a plurality of wheels mounted thereon.

9. A food service cart as defined in claim 3 wherein said cart includes a pair of doors and said switch means includes a pair of switches respectively associated with each door for activating said source of power when both doors are closed.

10. A food service cart as defined in claim 9 wherein said UV light source includes at least two UVC bulbs respectively mounted on the inner surfaces of said doors.

11. A food service cart as defined in claim 10 wherein said doors have recesses formed on their inner surfaces and said bulbs are mounted in said recess.

12. A food service transport cart comprising a mobile housing including at least one door providing selective access to the interior of the housing; means in the housing for supporting a plurality of food service trays; at least one UV light source mounted in said housing; a power supply source mounted in the housing for selectively supplying electrical power to the light source; switch means connected between said light source and said power supply source and located in said housing for contacting said door when the door is closed to allow electrical power to pass from said power supply source to said light source; and a timer connected to said switch and light source to cut off supply of electrical power to the light source after a predetermined period of time from closure of said door.

13. A food service transport cart as defined in claim 12 wherein said UV light source is mounted on the inside of said at least one door.

14. A food service transport cart as defined in claim 12 wherein said source of power is a battery pack.

15. A food service transport cart as defined in claim 13 wherein said source of power is a rechargeable battery pack.

16. A food service cart as defined in claim 12 wherein said cart includes a pair of doors and said switch means includes a pair of switches respectively associated with each door for activating said source of power when both doors are closed.

17. A food service cart as defined in claim 16 wherein said UV light source includes at least two UVC bulbs respectively mounted on the inner surfaces of said doors.

18. A food service cart as defined in claim 17 wherein said doors have recesses formed on their inner surfaces and said bulbs are mounted in said recess.

19. A food service cart as defined in claim 18 wherein said cart includes a plurality of wheels mounted thereon.

20. A food service cart as defined in claim 12 wherein said light source is located to directly irradiate all tray levels in the cart.
